(19) United States
(12) Patent Application Publication
(10) Pub. No.: US 2023/0277100 A1
(43) Pub. Date: Sep. 7, 2023

(54) OXIMETRY MONITORING IN A WEARABLE MEDICAL DEVICE

(71) Applicant: West Affum Holdings DAC, Dublin 2 (IE)

(72) Inventors: Dallas Eugene Meeker, Kirkland, WA (US); Joseph L. Sullivan, Kirkland, WA (US); Jaeho Kim, Redmond, WA (US); Kelly A. Brennan, Renton, WA (US)

(21) Appl. No.: 18/177,736

(22) Filed: Mar. 2, 2023

Related U.S. Application Data
(60) Provisional application No. 63/315,961, filed on Mar. 2, 2022.

(51) Int. Cl. A61B 5/1455; A61B 5/00; A61B 5/256; A61B 5/0205; A61B 5/026; A61N 1/39; A61B 5/28; A61B 5/33

(52) U.S. Cl. CPC A61B 5/14552; A61B 5/6823; A61B 5/256; A61B 5/0205; A61B 5/026; A61N 1/3904; A61B 5/28; A61B 5/33

(57) ABSTRACT

Technologies and implementations for facilitating oximetry monitoring in a wearable medical device (WMD). The oximetry monitoring may include pulse oximeter sensors of reflectance type and transmittance type. The technologies and implementations may include interactive devices and prompts to facilitate the oximetry monitoring.

### CROSS-REFERENCE TO RELATED APPLICATIONS

This application claims benefit of priority to U.S. Provisional Patent Application Ser. No. 63/315,961, filed Mar. 2, 2022, titled OXIMETRY MONITORING IN A WEARABLE MEDICAL DEVICE, which is incorporated herein by reference in its entirety for all purposes. This application may be related to U.S. patent application Ser. No. 17/133,939, filed on Dec. 24, 2020, titled: Wearable Medical Device with Integrated Blood Oxygen Saturation Level Device, which is incorporated herein by reference in its entirety for all purposes.

### BACKGROUND

Unless otherwise indicated herein, the approaches described in this section are not prior art to the claims in this application and are not admitted to be prior art by inclusion in this section.

Technology has contributed to improvements in healthcare. Some examples include healthcare related devices that may be mobile and personal. Mobile and personal healthcare devices may include Wearable Medical Devices (WMDs). Some WMDs may include medical devices that facilitate monitoring of various health related activities of a person. For example, a WMD may include a medical device that may be used to monitor a person's heart activity. The heart activity monitored by the WMD may be in the form of electrical signals (i.e., electrocardiogram or ECG). The WMD may be in a form factor capable of being worn by a person, whose heart activity is to be monitored. Monitoring of a person's ECG may facilitate intervention of heart related issues.

An example of a WMD, which may be used to monitor and facilitate intervention of a person's heart activity, may be a cardioverter defibrillator type medical device (e.g., wearable cardioverter defibrillator or WCD). Some examples of WCDs may include garment or some sort of support structure that a person may wear having electronic components and electrodes configured to facilitate monitoring the person's heart activity and to facilitate providing an electrical shock to the person's heart when treatment is necessary.

In addition to monitoring a person's heart, monitoring oxygen in a person's blood may help facilitate to determine the person's health. An example of monitoring the oxygen in a person's blood may be monitoring blood oxygen saturation levels. Monitoring the blood oxygen saturation levels may help facilitate monitoring of different health related conditions (e.g., vital signs including the heart rhythm, respiration, etc.). For example, blood oxygen saturation levels may affect various organ function such as, but not limited to, brain and heart. For example, low levels of blood oxygen saturation levels may lead to heart issues including cardiac arrest. Accordingly, monitoring blood oxygen saturation levels may help facilitate detection of a variety of health related issues.

Accordingly, health care devices having a variety of monitoring devices may be capable of addressing a variety of health related issues. The variety of monitoring devices may complement each other and may provide a more comprehensive indication of a person's health. These monitoring devices may be included in a WMD.

All subject matter discussed in this section of this document is not necessarily prior art and may not be presumed to be prior art simply because it is presented in this section. Plus, any reference to any prior art in this description is not and should not be taken as an acknowledgement or any form of suggestion that such prior art forms parts of the common general knowledge in any art in any country. Along these lines, any recognition of problems in the prior art are discussed in this section or associated with such subject matter should not be treated as prior art, unless expressly stated to be prior art. Rather, the discussion of any subject matter in this section should be treated as part of the approach taken towards the particular problem by the inventor(s). This approach in and of itself may also be inventive. Accordingly, the foregoing summary is illustrative only and not intended to be in any way limiting. In addition to the illustrative aspects, embodiments, and features described above, further aspects, embodiments, and features will become apparent by reference to the drawings and the following detailed description.

### SUMMARY

Described herein are various illustrative methods, systems, and apparatus for facilitating oximetry monitoring in a wearable medical device (WMD).

Some example systems include a support structure configured to be worn by a person. Some example systems include a monitoring component attached to the support structure; the monitoring component configured to provide information indicative of a cardiac condition of the person while the person is wearing the support structure. The example systems include an alert button, an oxygenation sensor, and a processor communicatively coupled to the alert button and the oxygenation sensor, the processor configured to cause an output to be issued to a user interface based, at least in part, on information received from the monitoring component, the output comprising a prompt to take an action using the alert button.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a wearable medical device (WMD), in accordance with various embodiments.

FIG. 2 illustrates a pulse oximeter sensor device integrated with an electrode, in accordance with various embodiments.

FIG. 3 illustrates a pulse oximeter sensor device integrated with an alert button, in accordance with various embodiments.

FIG. 4 illustrates a pulse oximeter sensor integrated with an alert button, in accordance with various embodiments.

FIG. 5 illustrates a WMD having a pulse oximeter sensor coupled with an alert button, in accordance with various embodiments.

FIG. 6 is a block diagram illustrating components of a defibrillator device, which may be used with various embodiments.

### DETAILED DESCRIPTION

The following description sets forth various examples along with specific details to provide a thorough understanding of claimed subject matter. It will be understood by those skilled in the art after review and understanding of the present disclosure, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to methods, systems, and apparatus for facilitating oximetry monitoring in a wearable medical device (WMD).

A wearable medical device (WMD) may be used to facilitate monitoring and treatment of various medical conditions of a person. In order to facilitate monitoring and treatment of medical conditions of a person, a WMD may be worn by the person. In order to help facilitate the wearing of the WMD, the WMD that may be included in a support structure configured to be worn by the person, where the support structure may include various components of the WMD. An example of a WMD that may facilitate monitoring and treatment of a person may include a WMD configured to facilitate monitoring and treatment of potential issues with a person's heart (i.e., the person may have a health condition, where the electrical control system of the heart may malfunction, which may cause the heart to beat irregularly or not at all). Commonly, these types of WMDs may include a defibrillator device.

Briefly, the above problem with the rate of the heartbeat may be generally referred to as arrhythmia. Arrhythmia may be caused by many factors, but in general, arrhythmia may be caused by a malfunction in the electrical control system of the heart. Some types of arrhythmias may result in inadequate blood flow resulting in reduction or lack of the amount of blood pumped to the various parts of the body. For example, issues with the sinoatrial (SA) node may lead to arrhythmia of some kind. Some arrhythmias may lead to a condition known as sudden cardiac arrest (SCA). In an SCA condition, the heart may fail to pump blood effectively, and as a result, death may occur.

An example type of arrhythmia, which may be associated with SCA, may be a condition known as ventricular fibrillation (VF). VF may be a condition where a ventricle or ventricles, which make up the heart to facilitate the pumping of blood, may make uncoordinated movements instead of steady rhythmic movements. In the VF condition, the heart may not pump adequate amounts of blood or may not pump blood at all, which may eventually lead to death. Another type of arrhythmia, which may be associated with SCA, may be a condition known as ventricular tachycardia (VT).

Turning back to WMDs, an electronic device may be utilized to help treat VF by defibrillating the heart. An example of this type of electronic device may be a defibrillator. A defibrillator may be capable of monitoring the electrical signals of the person's heart, and if necessary, administer treatment to the heart in the form of an electric shock. The defibrillator may monitor the electrical signals and provide the electric shock to the heart externally (i.e., through the surface of a body) via accessories commonly known as electrodes. The defibrillator may be in the form of a cardioverter defibrillator. As alluded to above, the cardioverter defibrillator may be included in a support structure configured to be worn by a person. (e.g., wearable cardioverter defibrillator or WCD), which may help facilitate monitoring the electrical activities of the person's heart and providing the electric shock to the heart in the VF condition. As a result, the WCD may help prevent Sudden Cardiac Death (SCD). The WCD may have a number of electrodes to facilitate monitoring of the electrical signals of the heart (e.g., rhythm of the heart) and a couple of electrodes to administer the electric shock as treatment. As part of the monitoring (e.g., arrythmia detection), the WCD may be configured to receive an electrocardiogram (ECG) signal from the number of electrodes (e.g., 5 ECG electrodes) on the skin of the person.

In accordance with various embodiments of the present disclosure, along with the monitoring of the person's heart, the person's blood oxygen saturation level may be monitored by including in the WMD an oxygenation sensor such as, for example, a pulse oximetry sensor to measure heart rate, blood flow, and blood oxygen saturation level. In some embodiments, an oxygenation sensor may include an optical sensor. As used herein, an oxygenation sensor may also be referred to as a pulse oximeter, a pulse oximetry device, an oximetry sensor or oximetry device. An oxygenation sensor may be but is not limited to, a reflective blood oxygen saturation level sensor. In some embodiments, the oxygenation sensor may be integrated with other components of a WMD such as, for example, electrodes and/or user interface components of the WMD. In some embodiments, the WMD comprises a WCD and uses the measured blood oxygenation saturation level in determining whether to provide shock and/or pacing therapy to the patient. In some embodiments of the above described WCD implementations, the WCD may include a wearable support structure (e.g., garment). This wearable garment, including the WCD, may be in a wide variety of forms such as, but not limited to, vests, shirts, undergarments, t-shirts, etc.

Additionally and/or alternatively, some non-limiting WMDs may include WMDs, which may be configured to be attachable on the skin of a person/patient. For example, a WMD may be “worn” by a person/patient by being attached to the skin rather than traditionally wearing a garment. Accordingly, the claimed subject matter is not limited in this respect. Some examples of a WMD configured to be worn utilizing adhesive methods may be found in published patent application US20210038156A1, tiled STABILIZING ACCESSORY FOR ADHESIVE MEDICAL DEVICES, which is incorporated by reference herein for all purposes.

As described above, the WCD may include a number electrodes to facilitate monitoring of electrical signals from the patient's heart and to facilitate an electric shock for the defibrillation process. Additionally, the WCD may include one or more electronic modules having many of the electronic components to facilitate monitoring and/or treatment of the heart (hereon, WCD monitor). The WCD monitor and the number of electrodes may facilitate the monitoring of the activities of the heart and the administration of the treatment of the heart (e.g., an electric shock for defibrillation, cardioversion and/or pacing). Accordingly, the number of electrodes may be disposed on the garment proximate to the patient's heart and/or close to or on the skin of the person. In accordance with the present disclosure, the WCD monitor may be disposed within and integrated with the garment resulting as a self-contained WCD.

The WCD monitor may comprise of various electronic components to facilitate operation of the WCD. For example, the WCD monitor may include a power supply such as, but not limited to, a battery to provide a defibrillator electrical shock to the patient via the electrodes. Along with the battery, the WCD monitor may include one or more capacitors as part of a discharge circuit for the shock. Additionally, the WCD monitor may include a user interface such as, but not limited to, a physical button (e.g., response buttons), graphical user interface (e.g., display, interactive and non-interactive), audible interface (e.g., indication sounds), etc. The operation and coordination of the electronic components may be facilitated by a processor included in the WCD monitor being communicatively coupled to the various electronic components to facilitate operation of the WCD. It should be appreciated after review of this disclosure that the above example components are just a few examples, and accordingly, electronic components of a WCD monitor may include a wide variety of electronic components to facilitate operation of the WCD. Additionally, some of the details of the WCD monitor will be described below.

Continuing with the non-limiting example of a WCD, the WCD may include blood oxygen saturation level sensors in accordance with various embodiments. The blood oxygen saturation level sensors may be implemented in a variety of methodologies such as, but not limited to, measurement of arterial oxygen saturation (SaO2) levels. Additionally, the SaO2 levels may be approximated by measurement of peripheral oxygen saturation (SpO2) levels, which may be measured using a pulse oximeter device. The sensors of the pulse oximeter device may be integrated with the garment of the WCD to facilitate utilization of the measurements of the blood oxygen saturation levels by the WCD.

In a non-limiting scenario of utilization of the disclosure, a person/patient (here on out, patient) may be wearing a WCD (i.e., the garment). A pulse oximeter sensor may be integrated with the garment. A WCD monitor may periodically indicate to the patient wearing the garment to use the pulse oximeter sensor to measure the patient's blood oxygen saturation level. The pulse oximeter sensor may be integrated into a portion of the garment (e.g., an over the shoulder strap). For example, the pulse oximeter sensor may be attached along a left shoulder strap allowing for the patient to use their right-hand finger for the pulse oximeter measurement by placing the finger on the pulse oximeter sensor. In one example, the pulse oximeter sensor may be discreetly integrated with the garment by having the various components of the pulse oximeter sensor recessed into the strap having a cover over the space configured to accommodate the finger. When prompted by WCD monitor, the patient may simply insert a finger into the space underneath the cover to activate the pulse oximeter sensor to take a measurement.

The prompting may be facilitated by a variety of methods. In one example, the prompting may be facilitated by an alert button. In this example, the alert button may be configured to be a user interface to facilitate interaction by the patient and/or a third party (e.g., medical personnel). Accordingly, the alert button may include a wide variety of interactable devices such as, but not limited, a physical person/patient input device that may be configured to be actuated, a graphical user interface that may be configured to be graphically interactable with a person/patient, and so forth. Accordingly, the claimed subject matter is not limited in this respect.

In one example, the pulse oximeter sensor may be integrated with the alert button, whereby the patient placing their finger on the alert button may cause the blood oxygen saturation level to be measured by the integrated pulse oximeter sensor. In this example, pulse oximeter sensor may be a transmittance type pulse oximeter sensor.

In another example, the prompting may be facilitated by a speaker. In this example, the speaker activated to provide an audio prompt for the patient to take a measurement of a blood oxygen saturation level. The audio prompt may include sounds and/or voice prompts, where the voice prompts may be interactive by guiding the patient.

In another example, the prompting may be facilitated by a graphical user interface. In this example, the graphical user interface may provide a visual prompt for the patient to take a measurement of a blood oxygen saturation level. The graphical user interface may be facilitated by a display. The display may be communicatively coupled with the WCD system. Accordingly, the display may be included in a separate device (e.g., a smartphone, smart watch, tablet, etc.).

In another non-limiting example scenario, the patient may be wearing the WCD. A pulse oximeter sensor may be of a reflectance type, where the pulse oximeter sensor may be configured to be in contact with the skin of the patient (i.e., adhering to the skin). In one example, the WCD may be configured to measure the blood oxygen saturation level at periodic intervals. In this example scenario, as the patient wears the WCD, the pulse oximeter sensor may become separated from the skin (i.e., the pulse oximeter sensor peeling off the skin). During one of the measurement periods, if a processor communicatively coupled with the WCD detects that the measurement of the blood oxygen saturation level may not be available (i.e., peeled off), the processor may be configured to issue a prompt to patient to adjust the pulse oximeter sensor or press the pulse oximeter sensor to ensure proper blood oxygen saturation level measurement.

In another non-limiting example scenario, the patient may be wearing the WCD. A pulse oximeter sensor may be of a reflectance type, where the pulse oximeter sensor may be configured to be included in one of the monitor electrodes. In this example scenario, the monitor electrode having the pulse oximeter sensor may become separated from the skin. Here again, the processor may be configured to issue a prompt to patient to adjust the monitor electrode or press the monitor electrode to ensure proper heart monitoring along with proper oxygen saturation level measurement.

In the last two non-limiting example scenarios, the patient may go about their day without actively taking steps to measure blood oxygen saturation level (i.e., passively measuring blood oxygen saturation level). The passive measurement of the blood oxygen saturation level may be periodically (e.g., hourly), at predetermined times (e.g., 9:00 a.m., 12:00 p.m., etc.), and/or substantially continuously.

In some examples, the functionality described may be facilitated by a processor included with the WMD (e.g., WCD monitor). In some examples, the functionality may be facilitated by a processor in a separate device (e.g., smartphone, tablet, etc.), where the smartphone may facilitate interaction with the WMD. Additionally, in some examples, the pulse oximeter sensor functionality may be provided by a separate device such as, but not limited to, a smart watch and/or smart band. Further, in some examples, the pulse oximeter sensor functionality may be provided by a pulse oximeter sensor patch, which may include communication capabilities to facilitate communication with the WMD.

One example of a wearable medical system (WMD) may be a wearable cardioverter defibrillator (WCD). Another example may be a wearable cardiac monitor, which can also be referred to as a wearable cardiac monitoring device. A pulse oximeter sensor may be included as part of a wearable component of a WMD system. For example, a pulse oximeter sensor may be configured to interface directly with the skin of a person, who may be wearing the WMD. The pulse oximetry sensor may be configured to sense signals at various predetermined time periods or substantially continuously, as the person wears the WMD system. Alternatively, measurement information received from a pulse oximetry sensor may be utilized to determine whether to trigger a response either by the person wearing the WMD and/or by a third person (e.g., medical personnel or emergency).

Some examples of pulse oximeter sensors may include reflective pulse oximetry sensors, where a reflective pulse oximeter sensor may be configured to contact the skin of the person. In this example, if the contact between the pulse oximeter sensor and the skin is not sufficient to measure the blood oxygen saturation level, a notification may be triggered (e.g., a “contact-off” signal) and may be processed by a processor communicatively coupled with the WMD, where the processor may be configured to issue a notification via an alert button for the person to adjust the pulse oximeter sensor on the skin or press the pulse oximetry sensor against the skin as the measurements are obtained.

In some embodiments, determining whether a rhythm is shockable may be further aided by the blood oxygen saturation level (e.g., blood flow, which may provide blood perfusion information). For example, in cases of perfusing ventricular tachycardia, the WCD may be configured to recognize the perfusion and not trigger an alarm and/or provide therapy (i.e., therapy shock). Along with heart rate and QRS width, blood oxygen saturation level (i.e., perfusion information) may aid in determinations related to shockable/non-perfusing heart rate ranges.

Determining blood oxygen saturation level (i.e., having a pulse oximeter sensor integrated with a WCD system) may facilitate detection of a wide variety of health-related conditions. For example, blood oxygen saturation level information may help detect non-perfusing cardiac arrest (e.g., heart failure condition). Additionally, blood oxygen saturation level information may help detect various respiration conditions such as, but not limited to, detection of sleep apnea. Further, blood oxygen saturation level may help in diagnosing new high or low blood pressure issues, as a person goes about their daily lives.

Integration of a pulse oximeter sensor with a WMD may facilitate obtaining blood oxygen saturation level from a person/a patient prescribed a WCD passively (i.e., without actions by the patient). When worn by the patient, the patient may be monitored by the WCD system, where the health-related information (e.g., obtaining blood oxygen saturation level along with heart related information) may be collected at some predetermined intervals, ad hoc, and/or substantially continuously. The collected health-related information may aid in determining various diagnosis including alerts. A patient developing a condition such as heart failure or a lung disease, which would otherwise require a doctor's office visit, may be monitored and diagnosed outside the doctor's office.

It should be appreciated after review of this disclosure that the above non-limiting examples facilitate utilization of a blood oxygen saturation level sensor with a WCD, where the blood oxygen saturation level sensor and the WCD may be integrated with a support structure. This integration may facilitate an increase in information of the health of a person through increased measurements such as, but not limited to, a person's ECG and a person's blood oxygen saturation level. Additionally, the increased information may facilitate identification of the person providing a more personal health monitoring system.

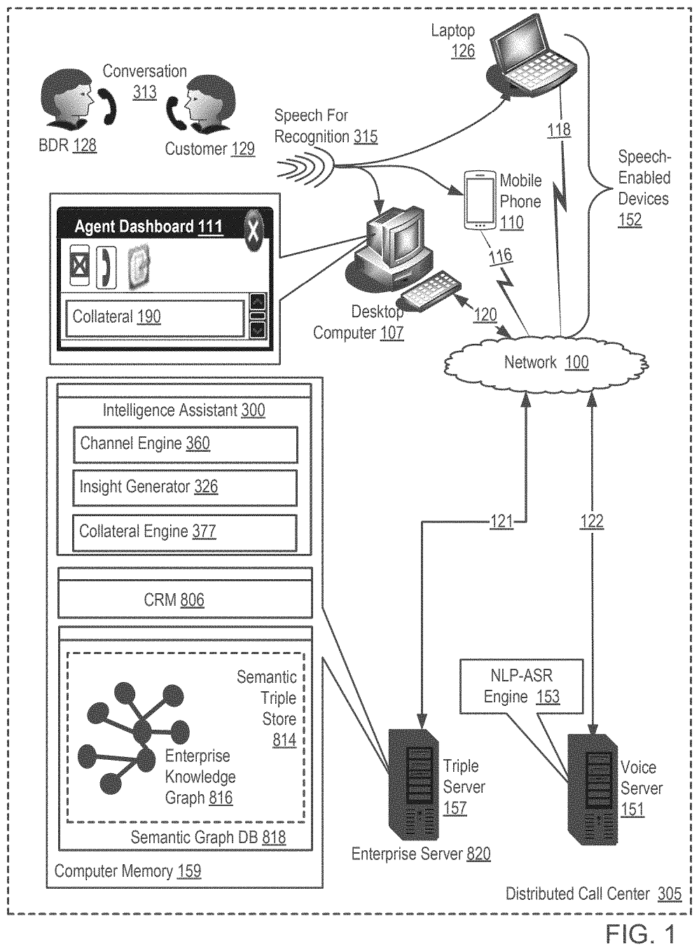

Turning now to FIG. 1, FIG. 1 illustrates a wearable medical device (WMD), in accordance with various embodiments. In FIG. 1, a WMD may be configured to facilitate monitoring and treatment of a person's heart such as, but not limited to, a wearable cardioverter defibrillator (WCD) 100. The WCD 100 may be included in a support structure (hereon, garment 102), which may be configured to be worn by a person 104. The garment 102 may include a wide variety of garments such as, but not limited to, a shirt, a vest, a belt, a jacket, a bra, a dress, etc., or other wearable structures such as for example a harness, an adhesive housing (e.g., similar to the housing disclosed in U.S. Pat. No. 8,024,037 or used in continuous glucose monitors), etc.

In some embodiments, the WCD may include various electronic components to facilitate the functionality of the WCD as a heart monitoring and defibrillator device. The various electronic components may be illustrated as a WCD module (hereon, a WCD monitor 106). The WCD 100 may include two therapy electrodes configured to defibrillate a person's heart 108, defibrillator electrodes 110 and a number of monitoring electrodes 112 configured to detect and measure the person's electrical heart activity (e.g., electrocardiogram or ECG). As shown, the monitoring electrodes 112 and the defibrillator electrodes 110 may be located proximate to the person's heart 108 and chest area. The monitoring electrodes 112 and the defibrillator electrodes may be communicatively coupled to the WCD monitor 106 via a number of electrical leads 114. Additionally, shown in FIG. 1, the garment 102 may include a monitoring device separate from the WCD monitor 106. The separate monitoring device may be a blood oxygen saturation level device having a sensor to measure the blood oxygen saturation level (hereon, an oximetry sensor 116). Oximetry sensor 116 can be used by monitor 106 to measure a blood oxygen saturation level of person 104. This blood oxygen saturation level can be used by monitor 106 to determine health aspects of person 104 with more accuracy. In some embodiments, oximetry sensor 116 may be integrated into the support structure 102. Accordingly, the WCD 100 may include measurements of the blood oxygen saturation level, which may facilitate an increase in accuracy of determining the health of the person, as previously described.

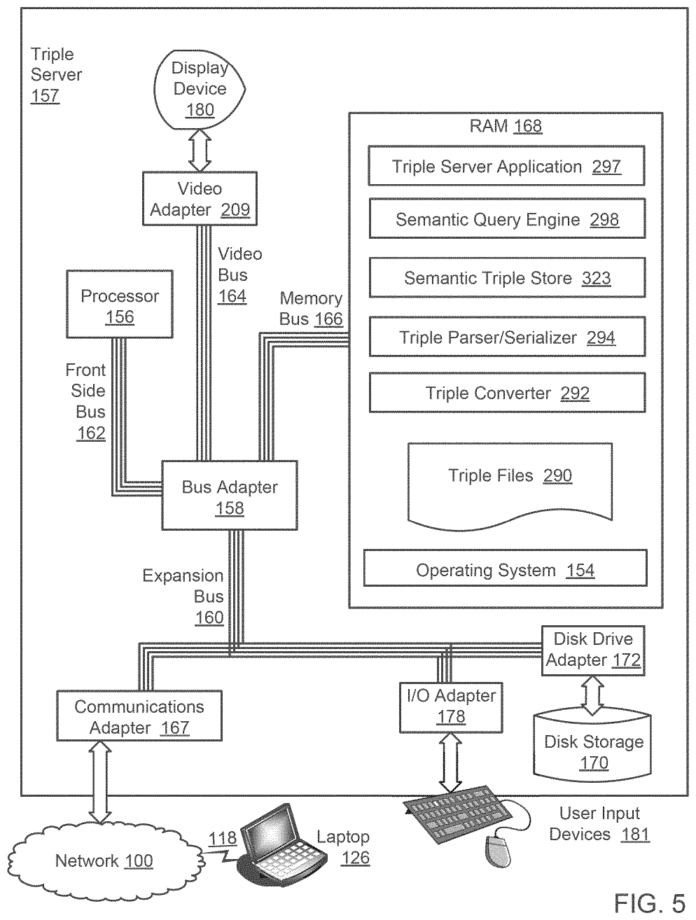

In other embodiments oximetry sensor 116 may be integrated in other components such as, for example, one of monitoring electrodes 112 or in an alert button (shown in FIG. 5). In embodiments, the alert button is configured to provide a user interface for person 104 to interact with WCD monitor 106. In some embodiments, the WCD 100 may prompt the person to press the alert button and/or operate oximetry sensor 116 and collect oximetry data.

As previously mentioned, the support structure 102 may be configured to support various components of the WCD 100, including an alert button, which may be communicatively coupled with the oximetry sensor 116. The WCD 100 may use measurements of the blood oxygen saturation level, which may facilitate an increase in accuracy of determining the health status of the person 104. In some embodiments, the alert button may prompt the person 104 to press the button and/or insert a fingertip and collect oximetry data. The alert button/transmissive oximetry sensor 116 combination may be attached to the outside of the person's clothing and facilitate access to respond to oximetry measurement prompts and/or any other prompt. In other embodiments, the oximetry sensor 116 may be a reflective sensor configured to be in direct contact with the skin or pressed against the skin when collecting blood oxygen saturation level measurements. If the contact is not adequate and/or proper, the alert button can be used to instruct the wearer to hold the sensor 116 against the skin of the person 104. In some embodiments, the alert button and the oximetry sensor 116 may be integrated into one housing. The housing combining both the alert button and the reflective oximetry sensor 116 may be configured to allow for the oximetry sensor 116 to directly contact the skin of the person 104. In an audible example, the button and a speaker may face outward and may be audible and easy to press against when needed. An alert button integrated with a reflective oximetry sensor may be inconspicuously hidden under clothing and secured in place. If the contact between the oximetry sensor 116 and the skin is not adequate and/or proper, the alert button may emit prompts to the person 104 to press the button/sensor. The person 104 may respond to a verbal prompt or vibration by pressing the button. If the contact between the oximetry sensor 116 and the skin is not adequate and/or proper, pressing of the button may cause the oximetry sensor 116 to be pressed against the skin and higher quality oximetry data may be collected.

In other embodiments of the WCD 100, WCD monitor 106 may include components configured to detect shockable heart rhythms and provide therapy such as pacing pulses and shocks such as defibrillation shocks and/or cardioversion shocks to the heart 108 of person 104. Such embodiments may be referred to herein as WCD embodiments of a WMD. Some WCD embodiments of a WMD may include two or more therapy electrodes 110 communicatively coupled to the WCD monitor 106 via one or more of electrical leads 114.

In one example, the oximetry sensor 116 may be communicatively coupled to the WCD monitor 106. In this example, the oximetry sensor 116 may have a sensor for measurements without the need for various other electronic components. Accordingly, the pulse oximeter device 116 may transmit blood oxygen saturation level measurements to the WCD monitor 106 to be processed and analyzed (e.g., utilize the processors and various electronic components of the WCD monitor 106 including its power supply). The processed and analyzed measurements may be provided to the person 104 via a display (not shown), which may be included in the WCD monitor 106.

In another example, the oximetry sensor 116 may be substantially self-contained (i.e., have various electronic components to function as a pulse oximeter device including a power supply). In this example, the oximetry sensor 116 may process and analyze the person's blood oxygen saturation level measurements and provide the results to the person 104 via a display, which may be included as part of the oximetry sensor 116.

In yet another example, the pulse oximeter device 116 and the WCD monitor 106 may be communicatively coupled, where the oximetry sensor 116 may process and analyze the person's blood oxygen saturation level measurements. The processed and analyzed measurements may be transmitted to the WCD monitor 106 to be displayed on the display on the WCD monitor 106.

In yet a further example, the oximetry sensor 116 and the WCD monitor 106 may be communicatively coupled to facilitate a hybrid self-contained oximetry sensor 116. In this example, the oximetry sensor 116 may configured to utilize one or more electronic components included in the WCD monitor 106. As a result, the WCD 100 may include a blood oxygen saturation level device (the oximetry sensor 116) to facilitate increased physiological parameters of the person's health contributing to an increase in accuracy of treatment (e.g., defibrillation) of the person 104.

In some embodiments, the oximetry sensor 116 may be integrated into a therapy electrode 110, while in some other embodiments, the oximetry sensor 116 may be integrated into the support structure 102, monitoring electrodes 112, or an alert button, as described above. In some embodiments, the oximetry sensor 116 may be coupled with a therapy electrode 110, while in some other embodiments, the oximetry sensor 116 may be physically coupled to the support structure 102, monitoring electrodes 112, or an alert button, as described above. For example, physically coupled utilizing an adhesive, a snap, a clasp, or other type of attachment. Some of these coupling methods may include the capabilities to be removable and/or reconfigurable by being attachable in other locations, which may facilitate improved sensing and data collection.

In some embodiments, the alert button and the oximetry sensor 116 may be coupled together by way of a snap or a pin through the support structure 102 and/or clothing, where the oximetry sensor 116 may be positioned on an inside of the support structure 102 to allow for the sensor be positioned against the skin, while the alert button may be on the outside of the support structure 102 and/or clothing to facilitate ease of access.

In some embodiments, the oximetry sensor 116 may be communicatively coupled to the WCD monitor 106. In this example, the oximetry sensor 116 may have a sensor for outputting sensed information to the WCD monitor 106 that can be used to determine blood oxygen saturation levels. In some embodiments, the oximetry sensor 116 may communicate blood oxygen saturation level data to the WCD monitor 106 to be processed and analyzed by a processor included in the WCD monitor 106 to determine blood oxygen saturation level measurements. The determined blood oxygen saturation level measurements may be provided to the person 104 via a display (not shown), which may be included in the WCD monitor 106 or a separate mobile device (not shown).

In another example, the oximetry sensor 116 may be substantially self-contained (i.e., have various electronic components including a sensor, a processor, and a power supply). In this example, the processor of the pulse oximeter device 116 may process and analyze the sensor output, determine the person's blood oxygen saturation level measurements, and provide the results to the person 104 via a display, which may be included as part of the pulse oximeter device 116.

As described with respect to the non-limiting scenarios, in one example, the WCD monitor 106, a mobile device app, and/or the alert button, may provide a prompt to the person 104 to measure a blood oxygen saturation level by utilizing the oximetry sensor 116, which may be configured to be attached to the alert button. In some embodiments, the person 104 may insert a fingertip into the oximetry sensor 116. The WCD monitor 106 may determine one or more prompts utilizing various factors such as, but not limited to, periodic schedule, detection of a low heart rate via the monitor electrodes 112, a predetermined change in the respiration rate, which may be determined by the WCD monitor 106, etc.

As previously described, WMD embodiments may comprise an oximetry sensor 116 combined or integrated together with an alert button, and worn as part of the wearable component of the WCD system. The WCD monitor 106 may be configured to use the alert button to issue a prompt to the person 104. The prompt may be configured to instruct the person 104 to measure a blood oxygen saturation level by utilizing the oximetry sensor 116 (e.g., the WCD monitor 106 detects an arrhythmia).

In yet another example, the oximetry sensor 116 may process, analyze, and display the person's blood oxygen saturation level measurements. The processed and analyzed measurements may also be transmitted to the WCD monitor 106 to be displayed on the display on the WCD monitor 106. Alternatively, or in addition, the measurements may be transmitted to a mobile device and displayed via an app on the mobile device.

In some embodiments, the WCD monitor 106 may include a defibrillation functionality as previously described and may issue such prompts in response to the detecting VF or a sustained VT and preparing to deliver a defibrillating electric shock to the person 104 via the therapy electrodes 110. The person 104 may respond by utilizing the oximetry sensor 116 (e.g., inserting a fingertip into the oximetry sensor 116, or clamping the oximetry sensor 116 onto the fingertip). In response to the person 104 utilizing the oximetry sensor 116, the WCD monitor 106 may terminate the shock process because utilization of the oximetry sensor 116 by the person 104 may be determined to indicate that the person 104 is conscious, and therefore not shockable.

In some embodiments, the oximetry sensor 116 may be configured to operate without the person 104 inserting or clamping a fingertip in the oximetry sensor 116. For example, the WCD monitor 106 may be configured to record additional blood oxygenation data from oximetry sensor 116 when the WCD monitor 106 detects a cardiac event. Such embodiments may be used as part of a WMD monitor alarm system. In some embodiments, oximetry sensor 116 may be implemented using a transmissive-type oximetry sensor such as, for example, a device that clamps to the earlobe of person 104. In other embodiments, oximetry sensor 116 can be implemented using a reflective-type oximetry sensor such as, but not limited to, a patch or the oximetry sensors used in some fitness trackers and smartwatches. Embodiments not requiring a user action, such as inserting or clamping of a fingertip, but instead collecting measurements without inconveniencing the wearer, may be advantageous when the person 104 is non-responsive. Because in these embodiments the oximetry sensor would not require an action from the patient (i.e., passive), the oximetry sensor may provide additional information as to whether the non-responsive person (e.g., unconscious) may be experiencing a perfusing or non-perfusing arrhythmia and determine its response based on the additional oximetry information.

Even though the WCD monitor 106 may be shown as a single module, the various cardiac monitoring components of the WCD 100 may be dispersed throughout the support structure 102. Additionally, the support structure 102 may be implemented as clothing such as, but not limited to, a vest, a jacket, a t-shirt, a dress shirt, a belt, a blouse, a coat, and any combination thereof. In some embodiments, the oximetry sensor 116 may be coupled to the alert button, and/or an electrode such as a therapy electrode 110 or an ECG electrode 112. In some embodiments, the oximetry sensor 116 may be coupled to other parts of the support structure 102 and/or a cardiac monitoring component such as an ECG electrode 112.

As previously described, oximetry sensory 116 can comprise a reflective oxygenation sensor. The reflective oxygenation sensor may be embedded in the support structure 102 (e.g., in parts of a garment or wearable components that are held against the skin of person 104 throughout the wear time). In other embodiments, the support structure 102 may include components that are temporarily biased against the skin of person 104 at times to collect the blood oxygen saturation level data. For example, in some embodiments oximetry sensor 116 comprises a reflective oxygenation sensor attached to or integrated with one of the monitoring electrodes 112 or the therapy electrodes 110. In such embodiments, the support structure 102 is configured to firmly hold the oximetry sensor and the electrodes against the skin of the patient to reduce motion at the electrode-skin interface for good sensing and/or therapy. The direct sensor to skin contact helps to obtain good quality oximetry data.

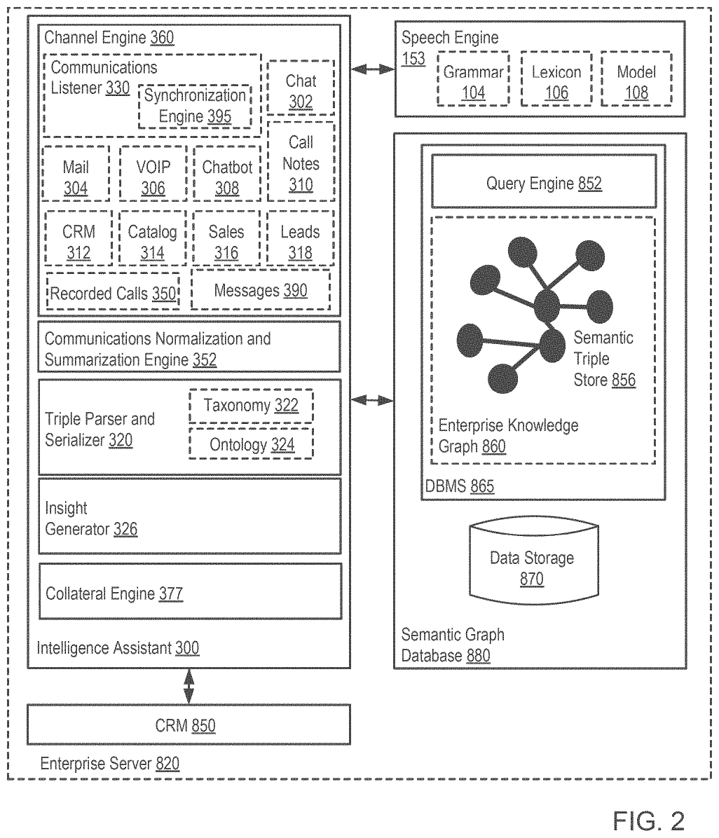

FIG. 2 illustrates a pulse oximeter sensor device integrated with an electrode, in accordance with various embodiments. In FIG. 2, an electrode assembly 200 may include an electrode 202 and a pulse oximeter 204. The electrode 202 may include a first surface 206 and a second surface 208. The first surface 206 may be a surface configured to attach to the skin of a person (e.g., receive electrical signals/ECG or provide therapy electrical signals/pacing and/or shock). The second surface 208 may be the outfacing surface of the electrode 202. As shown in FIG. 2, the first surface 206 may have integrated the pulse oximeter 204. Accordingly, when attached to the skin of the person, the electrode assembly 200 may be configured to either provide therapy (i.e., therapy electrode) or receive ECG signals (i.e., monitor electrodes) and facilitate measurement of the blood oxygen saturation level of the person (i.e., pulse oximeter sensor).

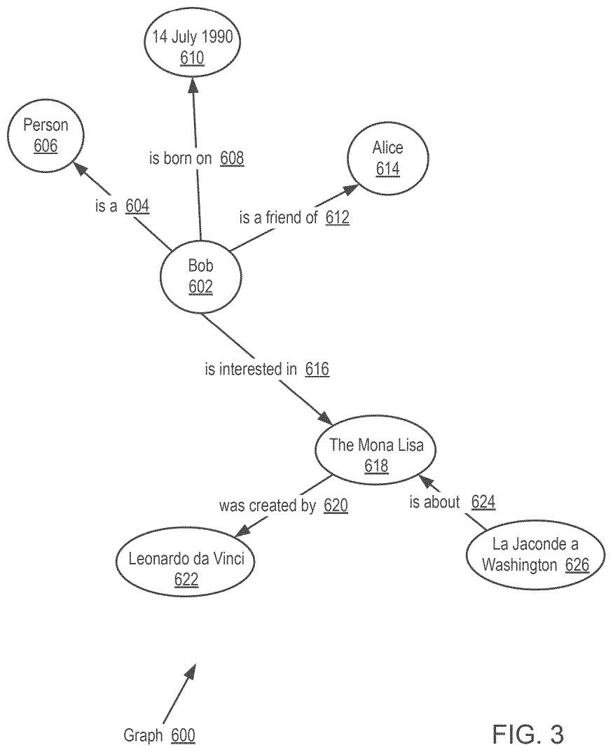

FIG. 3 illustrates a pulse oximeter sensor device integrated with an alert button, in accordance with various embodiments. In FIG. 3, an alert button assembly 300 may include an alert button 302 and a pulse oximeter sensor 304. As shown, the alert button assembly 300 may have a first side 306 and a second side 308. The first side 306 may include the alert button 302 having the pulse oximeter sensor 304. In one example, the alert button assembly 300 may be configured to facilitate actuation of the alert button 302 on the first side 306, and when the alert button 302 is actuated, the pulse oximeter sensor 304 may facilitate measurement of a blood oxygen saturation level.

In another example, the alert button assembly 300 may be held in place by a support structure such as, but not limited to, the support structure 102 shown in FIG. 1. The alert button assembly 300 may be held in such a manner to have the first side 306 held against the skin of a person. In this example, the pulse oximeter sensor 304 may be capable of facilitating measurement of the blood oxygen saturation level periodically and/or ad hoc (i.e., on demand). Here again, if contact with the skin separates to a level, where the blood oxygen saturation level may be negatively affected, the patient and/or the third party may be prompted to press the alert button 302 or press the second side 308. Pressing the second side 308 may press the first side 306 against the skin, which may cause the alert button 302 to actuate and/or improve skin contact for the pulse oximeter sensor 304. The prompt may include a specified duration to ensure that the skin contact with the pulse oximeter sensor 304 may be sufficient to facilitate the measurement, such as, but not limited to, SpO2 measurements.

As may be appreciated, the pulse oximeter sensor 304 shown in FIG. 3, may be of the reflectance type. In other embodiments, a transmissive-type oxygenation sensor may be used.

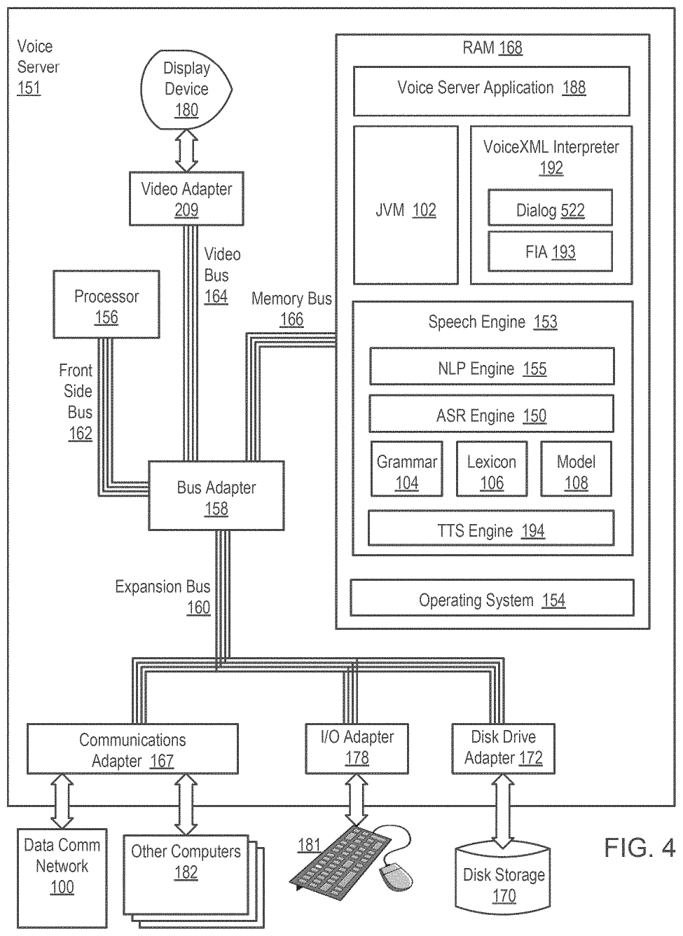

FIG. 4 illustrates a pulse oximeter sensor integrated with an alert button, in accordance with various embodiments. In FIG. 4, an alert button 402 may include a housing 404, and a pulse oximeter sensor 406, and a speaker 408. Additionally, the housing 404 may include a passage 410 configured to facilitate entry of a finger 412.

In FIG. 4, a person may be prompted to take a blood oxygen saturation level measurement. Responsive to the prompt, the person may place their finger 412 into the passage 410, where the pulse oximeter sensor 406 may facilitate measurement of the blood oxygen saturation level of the person.

In some embodiments, the alert button 402 may include the speaker 408 and/or vibration functionality, which may be used by the WMD to provide prompts to the person wearing the WMD to collect information from pulse oximeter sensor 406 such as, but not limited to, SpO2. Additionally, in some embodiments the WMD may use the speaker 408 and/or vibration functionality to provide feedback to the person wearing the WMD when one or more actions by the person are needed. Such actions may include the person actively doing something or the person pausing to not do anything, or the person staying still, etc. during the time when data may be collected. Once the data has been collected, the person may resume normal activity. In some embodiments, alert button 402 may include a visual interface (not shown), which may be configured to show the measurement of the blood oxygen saturation level. As previously described the pulse oximeter sensor 406 may be of a transmittance type.

FIG. 5 illustrates a WMD having a pulse oximeter sensor coupled with an alert button, in accordance with various embodiments. In FIG. 5, components of a WMD 500 may include a hub 502, three therapy electrodes 504, ECG sensors 512, and a user interface 506 (e.g., an alert button) with a pulse oximeter sensor 508. As shown in FIG. 5, the hub 502 may be communicatively coupled to the user interface 506 and a pulse oximetry sensor 508 by cables 510. Additionally, the hub 502 may be communicatively coupled to ECG electrodes 512 and to the three therapy electrodes 504. Further, the hub 502 may be communicatively coupled to a WMD monitor (e.g., the WCD monitor 106 shown in FIG. 1) via cable 511.

In some embodiments, the hub 502 may be removably attached to a receptacle (not shown) in a support structure (not shown) of the WMD. The receptable in the support structure may include electrical contacts that may be electrically coupled to ECG electrodes 512 that may be integrated into the support structure. In such embodiments, the hub 502 may serve as a connector that may physically connect (i.e., plug into) with receptacle to electrically connect to the ECG electrodes 512.

In some embodiments, the user interface 506 may be housed with pulse oximeter sensor 508. In some other embodiments, the pulse oximeter sensor 508 may be replaced with a pulse oximeter sensor such as shown in FIGS. 3 and/or 4. For example, the pulse oximeter sensor 508 may be of a transmittance type pulse oximeter sensor, which may require the person to insert a fingertip as shown in FIG. 4. Alternatively, the pulse oximeter sensor may be of reflectance type pulse oximeter sensor, which may be coupled to the back of the alert button's housing and configured to sense tissue oxygenation without requiring an action by the person wearing the WMD.

In some embodiments, the user interface 506 may be configured to instruct the wearer on how to collect pulse oximetry data. For example, the user interface 506 may include a processor or be under the control of a processor, where the processor may be configured to determine when to issue instructions to the person wearing the WMD to press a button of the user interface 506. Additionally, the processor may be configured to instruct the person to press against the person's skin and hold it for a predetermine period of time and/or issue instructions to the person to insert a fingertip into the pulse oximeter 508.

As previously described, the pulse oximeter sensor may be a reflectance type or transmittance type based, at least in part, on its application and/or configuration.

In some embodiments, various optional sensors (e.g., impedance sensors, motion sensors, non-invasive blood pressure sensors, etc.) may be communicatively coupled to the hub 502. The hub 502 may include various electronic components such as, but not limited to, circuitry for filtering and/or analog-to-digital conversion of ECG and/or other sensor output signals. As previously mentioned, the WMD may include the components of FIG. 5 along with a support structure similar to support structure 102 described in FIG. 1.

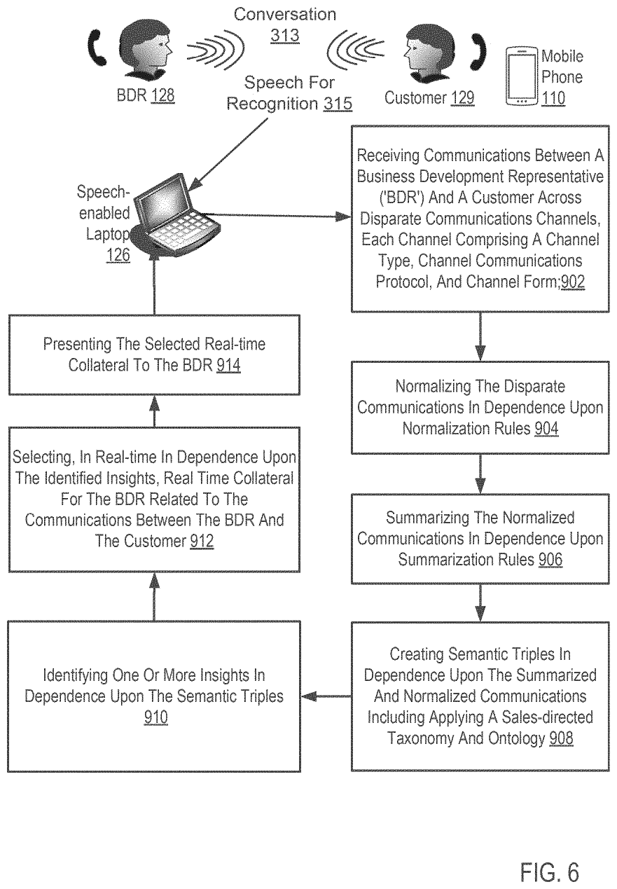

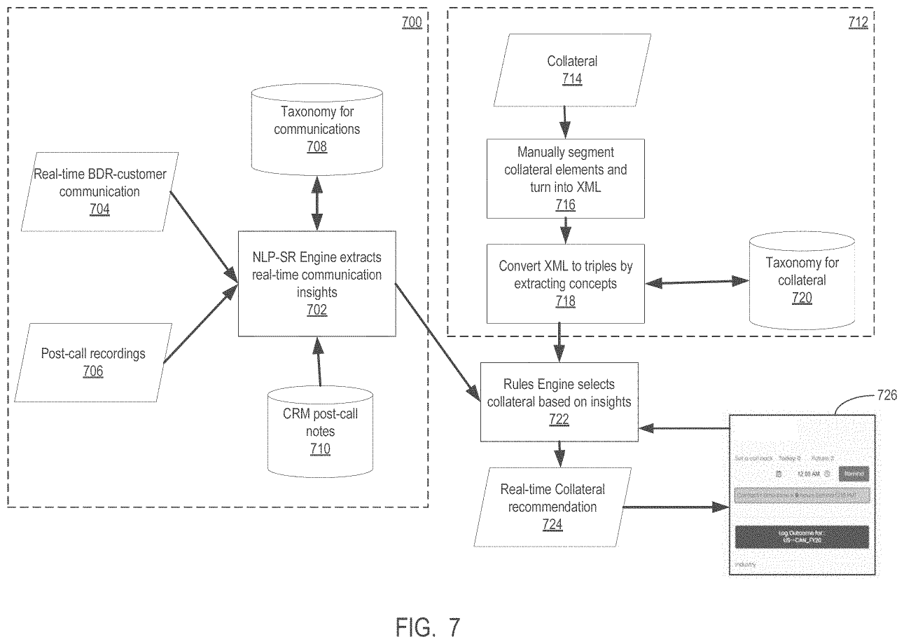

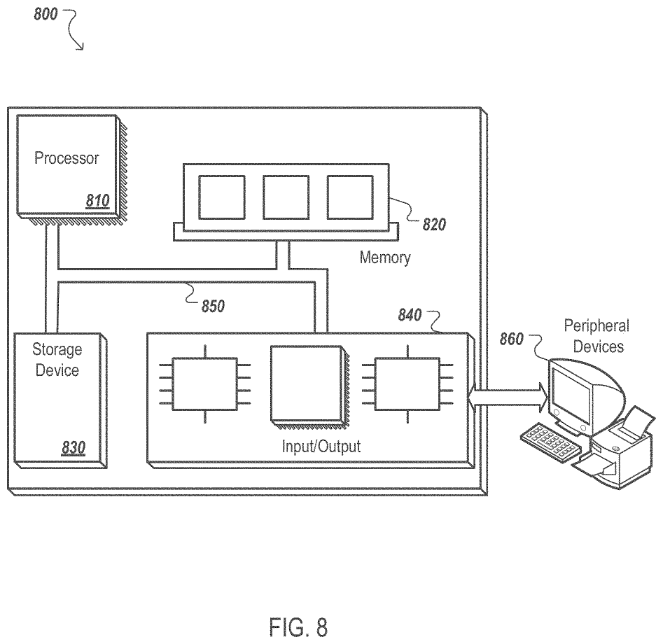

FIG. 6 is a block diagram illustrating components of a defibrillator device, which may be used with various embodiments. These components may be, for example, components of a WMD 100 and 500 (shown in FIGS. 1 and 5).

The defibrillator device 600 may be some of the above examples of one or more modules for the WCD (e.g., WCD monitor 106 shown in FIG. 1) intended for use by a user 680 (e.g., a wearer or person/patient 104 shown in FIG. 1). The defibrillator device 600 may typically include a defibrillation port 610, such as a socket in housing 601. The defibrillation port 610 may include nodes 614 and 618. One or more electrodes 604 and 608, which may be plugged into the defibrillation port 610, so as to make electrical contact with nodes 614 and 618, respectively. It may also be possible that the electrodes 604 and 608 may be connected continuously to the defibrillation port 610, etc. Either way, the defibrillation port 610 may be used for guiding via the electrodes 604 and 608 to a person 604 an electrical charge that may have been stored in the defibrillator device 600, as described herein.

The defibrillator device 600 may also have an ECG port 619 in the housing 601, for receiving ECG cables 609. The ECG cables 609 may facilitate sensing of an ECG signal (e.g., a 12-lead signal or from a different number of lead signals). Moreover, a defibrillator-monitor could have additional ports (not shown), and the other component 625 may be configured to filter the ECG signal (e.g., application of at least one filter to the signal to help facilitate removal of artifacts such as, but not limited to, chest compression due to chest compressions being delivered to the person).

The defibrillator 600 also may include a measurement circuit 620. The measurement circuit 620 may receive physiological signals from the ECG port 619, and also from other ports, if provided. The circuit 620 may render detected physiological signals and their corresponding information. The information may be in the form of data, or other signals, etc.

If the defibrillator 600 is configured as a WCD type device as described herein, ECG port 619 may not be present. The measurement circuit 620 may obtain physiological signals through the nodes 614 and 618 instead, when the electrodes 604 and 608 are attached to the person 604. In these cases, a person's ECG signal may be detected as a voltage difference between the electrodes 604 and 608. Additionally, the impedance between the electrodes 604 and 608 may be detected, among other things, whether the electrodes 604 and 608 have been inadvertently disconnected from the person.

The defibrillator 600 may also include a processor 630. The processor 630 may be implemented in a wide variety of manners for causing actions and operations to be performed. Some examples may include digital and/or analog processors such as microprocessors and digital-signal processors (DSPs), controllers such as microcontrollers, software running in a machine environment, programmable circuits such as Field Programmable Gate Arrays (FPGAs), Field-Programmable Analog Arrays (FPAAs), Programmable Logic Devices (PLDs), Application Specific Integrated Circuits (ASICs), and so on or any combination thereof.

The processor 630 may include a number of modules. One example module may be a detection module 632, which may detect outputs from the measurement circuit 620. The detection module 632 may include a VF detector. Accordingly, the person's detected ECG may be utilized to help determine whether the person is experiencing ventricular fibrillation (VF).

In another example module may be an advice module 634, which may provide advice based, at least in part, on outputs of detection module 632. The advice module 634 may include an algorithm such as, but not limited to, Shock Advisory Algorithm, implement decision rules, and so on. For example, the advice may be to shock, to not shock, to administer other forms of therapy, and so on. If the advice is to shock, some defibrillator examples may report the advice to the user, and prompt them to do it. In other examples, the defibrillator device may execute the advice by administering the shock. If the advice is to administer CPR, the defibrillator 600 may further issue prompts for administrating CPR, and so forth.

The processor 630 may include additional modules, such as module 636 for various other functions. Additionally, if other component 625 is provided, it may be operated in part by processor 630, etc. Other components 625 may include measurement of blood oxygen saturation level functionality as described herein.

In an example, the defibrillator device 600 may include a memory 638, which may work together with the processor 630. The memory 638 may be implemented in a wide variety of manners. For example, the memory 638 may be implemented such as, but not limited to, nonvolatile memories (NVM), read-only memories (ROM), random access memories (RAM), and so forth or any combination thereof. The memory 638 may include programs for the processor 630, and so on. The programs may include operational programs execution by the processor 630 and may also include protocols and methodologies that decisions may be made by advice module 634. Additionally, the memory 638 may store various prompts for the user 680, etc. Moreover, the memory 638 may store a wide variety of information (i.e., data) such as, but not limited to, information regarding the person.

The defibrillator 600 may also include a power source 640. In order to facilitate portability of defibrillator device 600, the power source 640 may include a battery type device. A battery type device may be implemented as a battery pack, which may be rechargeable or not rechargeable. At times, a combination of rechargeable and non-rechargeable battery packs may be utilized. Examples of power source 640 may include AC power override, where AC power may be available, and so on. In some examples, the processor 630 may control the power source 640.

Additionally, the defibrillator device 600 may include an energy storage module 650. The energy storage module 650 may be configured to store some electrical energy (e.g., when preparing for sudden discharge to administer a shock). The energy storage module 650 may be charged from the power source 640 to an appropriate level of energy, as may be controlled by the processor 630. In some implementations, the energy storage module 650 may include one or more capacitors 652, and the like.

The defibrillator 600 may include a discharge circuit 655. The discharge circuit 655 may be controlled to facilitate discharging of the energy stored in energy storage module 650 to the nodes 614 and 618, and also to electrodes 604 and 608. The discharge circuit 655 may include one or more switches 657. The one or more switches 657 may be configured in a number of manners such as, but not limited to, an H-bridge, and so forth.

The defibrillator device 600 may further include a user interface 670 for the user 680. The user interface 670 may be implemented in a variety of manners. For example, the user interface 670 may include a display screen capable of displaying what is detected and measured, provide visual feedback to the user 680 for their resuscitation attempts, and so forth. The user interface 670 may also include an audio output such as, but not limited to, a speaker to issue audio prompts, etc. The user interface 670 may additionally include various control devices such as, but not limited to, pushbuttons, touch display, and so forth. Additionally, the discharge circuit 655 may be controlled by the processor 630 or directly by the user 680 via the user interface 670, and so forth.

Additionally, the defibrillator device 600 may include other components. For example, a communication module 690 may be provided for communicating with other machines and/or the electrodes. Such communication may be performed wirelessly, or via wire, or by infrared communication, and so forth. Accordingly, information may be communicated, such as personal data, incident information, therapy attempted, CPR performance, ECG information, and so forth.

It should be appreciated after review of this disclosure that it is contemplated within the scope and spirit of the present disclosure that the claimed subject matter may include a wide variety of healthcare devices. Accordingly, the claimed subject matter is not limited in these respects.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter is not limited to the particular examples disclosed, but that such claimed subject matter also may include all implementations falling within the scope of the appended claims, and equivalents thereof.

1. A wearable medical device (WMD), comprising:
a support structure configured to be worn on a torso by a person;
a monitoring component configured to be coupled to the support structure, the monitoring component further configured to sense one or more physiological parameters of the person and provide information indicative of a cardiac condition of the person while the person is wearing the support structure;
an interactive device, the interactive device configured to receive an input from the person;
an oxygenation sensor, wherein the oxygenation sensor and the interactive device comprise an integrated unit; and
a processor communicatively coupled to the interactive device and the oxygenation sensor, the processor configured to cause an output to be issued to a user interface based, at least in part, on information received from the monitoring component, the output comprising a prompt to take an action using the interactive device.

2. The WMD of claim 1, wherein the oxygenation sensor comprises a reflective oxygenation sensor.

3. The WMD of claim 1, wherein the monitoring component comprises one or more components configured to sense one or more blood flow parameters and the processor is further configured to determine a trend of blood flow data from the sensed one or more blood flow parameters.

4. The WMD of claim 1, wherein the monitoring component comprises an electrocardiogram (ECG) electrode.

5. The WMD of claim 1, wherein the monitoring component comprises a therapy electrode.

6. The WMD of claim 5, wherein the processor is further configured determine whether the person should be provided therapy using the therapy electrode based, at least in part, on the information received from the monitoring component and information received from the oxygenation sensor.

7. The WMD of claim 1, wherein the interactive device comprises a button and the oxygenation sensor is integrated with the button so that operation of the oxygenation sensor actuates the button.

8. The WMD of claim 1, wherein the oxygenation sensor is configured to cause the interactive device to indicate an input from the person in response to the oxygenation sensor being operated by the person.

9. The WMD of claim 1, wherein the oxygenation sensor is disposed on a shoulder strap of the support structure.

10. A wearable medical device (WMD), comprising:
a support structure configured to be worn on a torso by a person;
an electrode coupled with the support structure;
an oxygenation sensor integrated with the electrode;
a processor communicatively coupled with the electrode and the oxygenation sensor, wherein the processor configured to continuously monitor an electrocardiogram (ECG) of the person received from the electrode while the person is wearing the support structure.

11. The WMD of claim 10, wherein the support structure is configured to position the oxygenation sensor and the electrode against a skin of the person while the person is wearing the support structure.

12. The WMD of claim 10, wherein the oxygenation sensor is configured to passively measure a blood oxygenation of the person while the person is wearing the support structure.

13. The WMD of claim 10, wherein the electrode comprises an ECG electrode and the oxygenation sensor comprises an optical sensor integrated in a face of the ECG electrode with no electrical connection between the ECG electrode and the oxygenation sensor.

14. The WMD of claim 10, wherein the processor is configured to continuously monitor blood oxygenation of the person using the oxygenation sensor.

15. The WMD of claim 10, wherein the processor comprises the processor configured to determine whether the person should be provided therapy based, at least in part, on the information received from the electrode and the oxygenation sensor.

16. A wearable cardioverter defibrillator (WCD) comprising:
a plurality of monitor electrodes, the plurality of monitor electrodes configured to receive electrocardiogram (ECG) from a person;
a plurality of therapy electrodes through which the WCD is configured to provide an electrical therapy to the person;
a WCD monitoring component coupled to the plurality of monitor electrodes and the plurality therapy electrodes, the WCD monitoring component configured to provide information indicative of a cardiac condition of the person while the person is wearing the support structure;
an interactive device, the interactive device communicatively coupled with the WCD monitoring component and configured to receive an input from the person;
an oxygenation sensor, the oxygenation sensor communicatively coupled with the WCD monitoring component and configured to passively measure a blood oxygenation of the person while the person is wearing the support structure;
a support structure configured to be worn by the person to position the plurality of monitor electrodes on a torso of the person and to position the oxygenation sensor against a skin of the person; and
a processor communicatively coupled with the WCD monitoring component and configured to cause an output to be issued to a user interface based, at least in part, on the information received from the monitoring component, the output comprising a prompt to take an action using the interactive device.

17. The WCD of claim 16, wherein the processor is configured to analyze the measured blood oxygenation in response to the information received from the WCD monitoring unit being indicative of an arrhythmia.

18. The WCD of claim 16, wherein the oxygenation sensor and the interactive device comprise an integrated unit.

19. The WCD of claim 16, wherein the oxygenation sensor is integrated with one of the plurality of monitor electrodes and is configured to continuously monitor blood oxygenation of the person.

20. The WCD of claim 16, wherein the processor is integrated with the WCD monitoring component.
